(19) United States
(12) Patent Application Publication
(10) Pub. No.: US 2025/0384976 A1
(43) Pub. Date: Dec. 18, 2025

(54) METHODS, SYSTEMS, AND COMPUTER PROGRAM PRODUCTS FOR GENERATING FOR IMPROVING MACHINE RECOGNITION OF RELATIONSHIPS BETWEEN MEDICAL ENTITIES

(71) Applicant: Optum, Inc., Minnetonka, MN (US)

(72) Inventors: Changsung Moon, Kirkland, WA (US); Feili Yu, Shoreline, WA (US)

(21) Appl. No.: 18/745,275

(22) Filed: Jun. 17, 2024

(51) Int. Cl. G16H 10/60; G16H 50/70

(52) U.S. Cl. CPC G16H 10/60; G16H 50/70

(57) ABSTRACT

A method includes receiving, a plurality of records containing clinical information associated with one or more patients; extracting, using a Natural Language Processing (NLP) model, a plurality of medical entities from the clinical information to generate a first data set that contains the plurality of medical entities; denoising, the first dataset to generate a second data set by: determining relationship strengths between pairs of respective ones of the medical entities; identifying a subset of the pairs of the respective ones of the plurality of medical entities that satisfy a relationship strength threshold; generating uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating a relevance score for each of the subset of pairs based on one or both of the uncommonality scores for the first and second ones of the medical entities included in the respective pair and a frequency of occurrence of the respective pair in the clinical information; and generating a knowledge graph data structure representing ones of the subset of pairs having relevance scores, respectively, that satisfy a relevance threshold.

### FIELD

The present disclosure relates generally to health care systems and services and, more particularly, to generation of knowledge graphs based on medical entities contained in clinical information.

### BACKGROUND

A knowledge graph is a semantic network that visualizes entities and the relationships between them. The information represented by the knowledge graph may be stored in a graph database. An entity is an object, such as an event, person, or thing. In a knowledge graph, these entities are represented as nodes. Each node/entity may be related to other nodes/entities. The relationships are represented by edges, which are connections between the nodes. Knowledge graphs may be applied to the field of healthcare services as a way to store and infer relationships between healthcare data or information and to improve the performance of predictive models, such as those provided through Artificial Intelligence or determinative models based on rules. Construction of a knowledge graph for a health care application, however, may be challenging due to a lack of a representative knowledge graph construction taxonomy. Example healthcare related knowledge graphs may be built by humans with domain knowledge of healthcare, but such a build approach may be slow and costly. Attempts to build a healthcare knowledge graph based on electronic health records associated with patients have been met with challenges due to the lack of a definitive mapping between clinical medical entities, such as drugs, diagnoses, procedures, and the like. For example, even though Drug A and Drug B appear in the same electronic health record, it may not be clear how these two drugs are related. Also, the appearance of Drug A and Disease C in an electronic health record does not necessarily mean that Drug A is being used to treat Disease C. A patient's chart or health record often includes multiple types of symptoms, diagnoses, and drugs. These vague relationships may make it difficult to build knowledge graphs from electronic health records.

### SUMMARY

According to some embodiments of the disclosure, a computer-implemented method comprises: receiving, by one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.

In other embodiments, the NLP model is a deep learning model.

In still other embodiments, determining the relationship strengths comprises: quantifying, by the one or more processors, the relationship strengths between the pairs of respective ones of the medical entities based on a frequency of occurrence of respective ones of the pairs in the clinical information.

In still other embodiments, the uncommonality score for the first one of medical entities is given by a log of a ratio of a size of the set of distinct names that the second medical entity can assume to a sum across the entire set of instances of the second medical entity in the clinical information of a first commonality factor; and the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.

In still other embodiments, the uncommonality score for the second one of medical entities is given by a log of a ratio of a size of the set of distinct names that the first medical entity can assume to a sum across the entire set of instances of the first medical entity in the clinical information of a second commonality factor; and the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.

In still other embodiments, the relevance score is given by a product of the uncommonality score for the first one of the medical entities and a log of the sum of one plus a number of times that the first medical entity occurs with the second medical entity in the clinical information across the entire set of instances of the second medical entity in the clinical information.

In still other embodiments, the relevance score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.

In still other embodiments, generating the knowledge graph comprises: generating, by the one or more processors, Resource Description Framework (RDF) triples based on the subset of pairs having relevance scores, respectively, that satisfy the relevance threshold; and configuring, by the one or more processors, the knowledge graph with the RDF triples.

In still other embodiments, the clinical information comprises patient health record information, medical claim information, or both the patient health record information and the medical claim information.

In some embodiments of the disclosure, a system comprises one or more processors and a memory coupled to the one or more processors and comprising computer readable program code embodied in the memory that is executable by the one or more processors to perform operations comprising: receiving, by one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.

In further embodiments, the NLP model is a deep learning model.

In still further embodiments, determining the relationship strengths comprises: quantifying, by the one or more processors, the relationship strengths between the pairs of respective ones of the medical entities based on a frequency of occurrence of respective ones of the pairs in the clinical information.

In still further embodiments, the uncommonality score for the first one of medical entities is given by a log of a ratio of a size of the set of distinct names that the second medical entity can assume to a sum across the entire set of instances of the second medical entity in the clinical information of a first commonality factor; and the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.

In still further embodiments, the uncommonality score for the second one of medical entities is given by a log of a ratio of a size of the set of distinct names that the first medical entity can assume to a sum across the entire set of instances of the first medical entity in the clinical information of a second commonality factor; and the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.

In still further embodiments, the relevance score is given by a product of the uncommonality score for the first one of the medical entities and a log of the sum of one plus a number of times that the first medical entity occurs with the second medical entity in the clinical information across the entire set of instances of the second medical entity in the clinical information.

In still further embodiments, the relevance score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.

In still further embodiments, generating the knowledge graph comprises: generating, by the one or more processors, Resource Description Framework (RDF) triples based on the subset of pairs having relevance scores, respectively, that satisfy the relevance threshold; and configuring, by the one or more processors, the knowledge graph with the RDF triples.

In still further embodiments, the clinical information comprises patient health record information, medical claim information, or both the patient health record information and the medical claim information.

In some embodiments of the disclosure, one or more a non-transitory computer readable storage media comprise computer readable program code embodied in the media that is executable by one or more processors to perform operations comprising: receiving, by one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.

In other embodiments, the relevance score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.

Other methods, systems, articles of manufacture, and/or computer program products according to embodiments of the disclosure will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an intelligent Decision Support System (DSS) for associating medical entities in a knowledge graph in accordance with some embodiments of the disclosure;

FIG. 2 is a block diagram of the intelligent DSS for associating medical entities in a knowledge graph in accordance with some embodiments of the disclosure;

FIG. 3 is a block diagram of a medical entity extraction system in accordance with some embodiments of the disclosure;

FIG. 4 is a flowchart that illustrates operations of the intelligent DSS for associating medical entities in a knowledge graph in accordance with some embodiments of the disclosure;

FIG. 5 illustrates generation of medical entity uncommonality scores in accordance with some embodiments of the disclosure;

FIG. 6 is a table that illustrates ranking of medications based on a relevance score with respect to a diagnosis;

FIG. 7 is a knowledge graph generated using an intelligent DSS according to some embodiments of the disclosure;

FIG. 8 is a data processing system that may be used to implement an intelligent DSS for associating medical entities in a knowledge graph in accordance with some embodiments of the disclosure; and

FIG. 9 is a block diagram that illustrates a software/hardware architecture for use in an intelligent DSS for associating medical entities in a knowledge graph in accordance with some embodiments of the disclosure.

### DETAILED DESCRIPTION

In the following detailed description, numerous specific details are set forth to provide a thorough understanding of embodiments of the disclosure. However, it will be understood by those skilled in the art that embodiments of the disclosure may be practiced without these specific details. In some instances, well-known methods, procedures, components, and circuits have not been described in detail so as not to obscure embodiments of the disclosure. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

As used herein a “procedure” may be, but is not limited to, any type of treatment provided by a provider to a patient or any type of medicine or product prescribed or given to a patient for treatment. In general, a “procedure” may be defined as any activity directed at or performed on an individual with the object of improving health, treating disease or injury, or making a diagnosis.

As used herein a medical entity may include, but is not limited to a disease (e.g., medical conditions and diagnoses), a medication (e.g., pharmaceutical drugs or drug therapies used for treatment), a procedure (e.g., diagnostic procedures, therapeutic procedures, and medical devices), lab (e.g., pathology and laboratory procedures), and a symptom (e.g., clinical signs and symptoms). Within the medication category, the following sub-categories may be used: a dosage (e.g., amount or strength of a drug including units), form (e.g., the physical form of a dose of drug when it is administered), and a route (e.g., the way in which a drug is taken into the body).

Embodiments of the disclosure are described herein in the context of a Decision Support System (DSS) that includes one or more Artificial Intelligence (AI) models for processing patient records, which include clinical information, and associating medical entities with one another using a knowledge graph. The one or more AI models of the intelligent DSS be embodied in a variety of different ways including, but not limited to, one or more of the following AI systems: a multi-layer neural network, a machine learning system, a deep learning system, a large language model, a natural language processing system, and/or computer vision system. Moreover, it will be understood that the multi-layer neural network is a multi-layer artificial neural network comprising artificial neurons or nodes and does not include a biological neural network comprising real biological neurons. The AI models described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the adjudication process and/or the AI training process to improve the efficiency of a computer system when the computer system operates in an inference mode to make an inference, prediction, classification, suggestion, or the like with respect to medical entities with each other in a knowledge graph.

Some embodiments of the disclosure stem from a realization that automated systems for generating a healthcare knowledge graph based on electronic health records involves counting clinical entity pairs, such as a count of specific diagnoses and medications appearing together. A relation may be quantified based on the percentage of this count over a total number of counts given the same diagnoses or given the same medication. Such a quantification is called conditional probability. Such an approach may suffer from inaccuracies due to the lack of a definitive one-to-one relationship between medical entities. Many common medical entities may be counted disproportionately more than other medical entities. For example, a flu vaccine may routinely be prescribed along with other drugs or medications, which may distort the true relationships between various medical entity pairs. Healthcare knowledge graphs that are built manually based on review by medical professionals may be of high quality, but may also be costly and of limited breadth, i.e., they may not cover as many possible relationships between medical entities as would be desirable. This may be because only proved relationships are provided in these knowledge graphs.

According to some embodiments of the disclosure an intelligent DSS that generates suggested pairings for associating medical entities in a knowledge graph is provided. The intelligent DSS receives one or more records containing clinical information associated with one or more patients. The clinical information in these records may be processed using one or more models including a medical entity extraction model, which is a deep learning based Named Entity Recognition (NER) model that is configured to extract medical entities from the clinical information. Relationship strengths between pairs of the medical entities may be determined based on the frequency of occurrence of the respective pairs in the clinical information. To remove noise (i.e., denoise) from the relationship pairs extracted from the clinical information, a statistical measure of uncommonality is defined for each medical entity. The more common a medical entity is; the lower the uncommonality score. A relevant score is generated that is based on both the uncommonality scores for the medical entities and the frequency the medical entity pairing occurs in the clinical information. A non-transitory computer readable medium is configured with a knowledge graph that contains those pairs of medical entities having relevance scores that satisfy a relevance threshold.

Advantageously, a knowledge graph configured in the non-transitory computer readable medium may provide an information dense compilation of health care information that is efficiently accessible using one or more processors in a variety of healthcare applications including, but not limited to, predictive modeling of diseases, care regiments, claim generation, and the like. Moreover, the accuracy of the knowledge graph may be improved by filtering out those medical entity relationships based on high frequency medical entities that are unlikely to have a relevant relationship with many or most other medical entities, e.g., many patients receive a flu shot, but this medication is mostly unrelated to other treatments or drugs the patients receive.

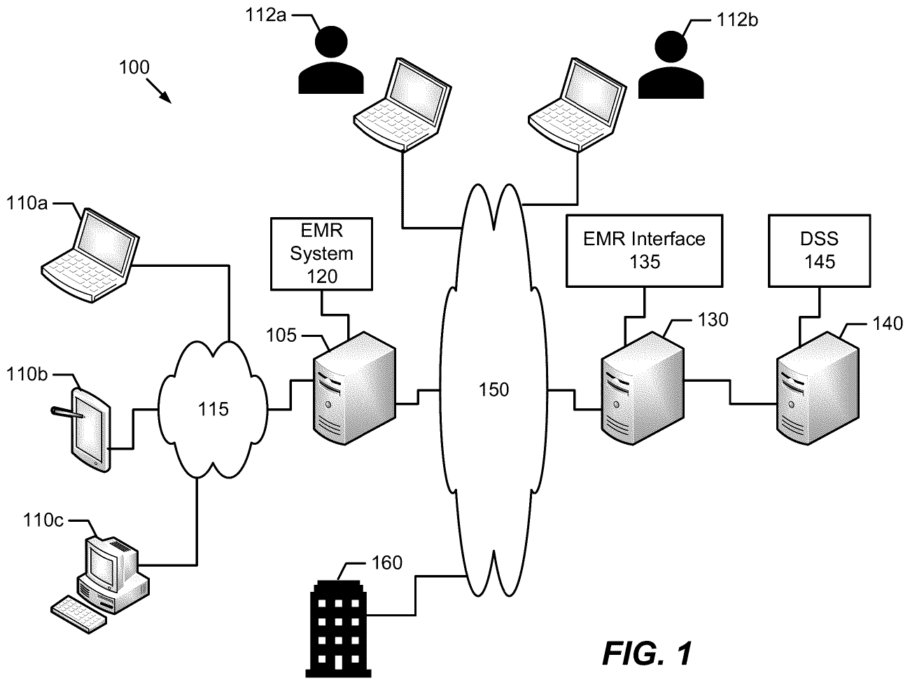

Referring to FIG. 1, a communication network 100 including an intelligent DSS for associating medical entities in a knowledge graph, in accordance with some embodiments of the disclosure, comprises a health care facility server 105 that is coupled to devices 110a, 110b, and 110c via a network 115. The health care facility may be any type of health care or medical facility, such as a hospital, doctor's office, specialty center (e.g., surgical center, orthopedic center, laboratory center etc.), or the like. The health care facility server 105 may be configured with an Electronic Medical Record (EMR) system module 120 to manage patient files and facilitate the entry of orders for patients via health care service providers (“providers”). Although shown as one combined system in FIG. 1, it will be understood that some health care facilities use separate systems for electronic medical record management and order entry management. The providers may use devices, such as devices 110a, 110b, and 110c to manage patients' electronic charts or records and to issue orders for the patients through the EMR system 120. An order may include, but is not limited to, a treatment, a procedure (e.g., surgical procedure, physical therapy procedure, radiologic/imaging procedure, etc.) a test, a prescription, and the like. The network 115 communicatively couples the devices 110a, 110b, and 110c to the health care facility server 105. The network 115 may comprise one or more local or wireless networks to communicate with the health care facility server 105 when the health care facility server 105 is located in or proximate to the health care facility. When the health care facility server 105 is in a remote location from the health care facility, such as part of a cloud computing system or at a central computing center, then the network 115 may include one or more wide area or global networks, such as the Internet. The providers may operate by providing health care services for patients and then invoicing one or more payors 160 for the services rendered. The payors 160 may include, but are not limited to, providers of private insurance plans, providers of government insurance plans (e.g., Medicare, Medicaid, state, or federal public employee insurance plans), providers of hybrid insurance plans (e.g., Affordable Care Act plans), providers of private medical cost sharing plans, and the patients themselves.

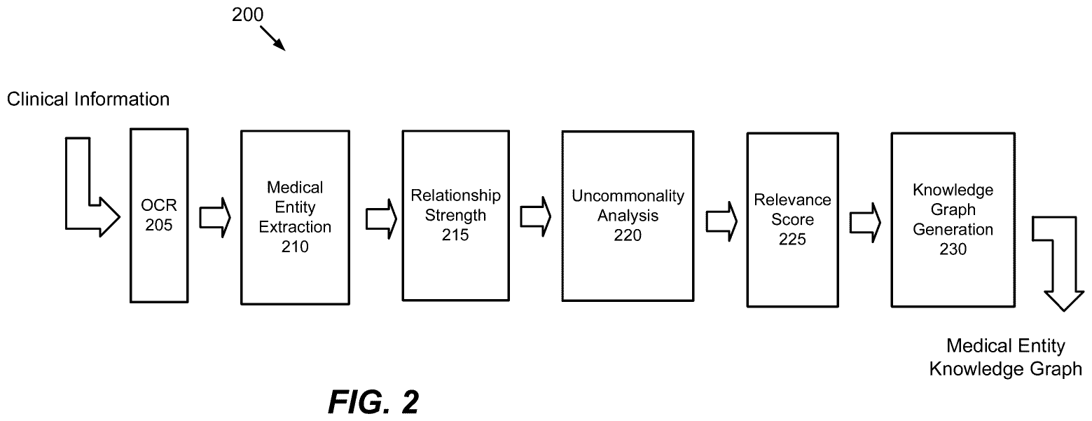

According to some embodiments of the disclosure, an intelligent DSS for associating medical entities in a knowledge graph may be provided to assist entities, such as providers, payors, auditors, data entry personnel, and others, which are represented as users 112a and 112b in FIG. 1, in processing one or more patient clinical records to associate medical entities in a knowledge graph. The intelligent DSS may include a health care facility interface server 130, which includes an EMR interface system module 135 to facilitate the transfer of information between the EMR system 120, which the providers use to manage patient charts and records and issue orders, and a knowledge graph generation server 140, which includes a DSS module 145. The knowledge graph generation server 140 and DSS module 145 may be configured to receive patient records from the EMR system 120 by way of the health care facility interface server 130 and EMR interface module 135. The knowledge graph generation server 140 and DSS module 145 may process each page of each patient clinical record using an AI supported DSS as will be described below with respect to FIG. 2 to generate medical entity pairing suggestions for one or more portions of the clinical information contained therein. A non-transitory computer readable medium may be configured with a knowledge graph including the suggested medical entity pairings.

It will be understood that the division of functionality described herein between the knowledge graph generation server 140/DSS module 145 and the health care facility interface server 130/EMR interface module 135 is an example. Various functionality and capabilities can be moved between the knowledge graph generation server 140/DSS module 145 and the health care facility interface server 130/EMR interface module 135 in accordance with different embodiments of the disclosure. Moreover, in some embodiments, the knowledge graph generation server 140/DSS module 145 and the health care facility interface server 130/EMR interface module 135 may be merged as a single logical and/or physical entity.

A network 150 couples the health care facility server 105, the health care facility interface server 130, the payor(s) 160, and the users 112a, 112b together. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The medical entity knowledge graph generation service provided through the health care facility interface server 130, EMR interface module 135, knowledge graph generation server 140 and DSS module 145 to associate medical entities in a knowledge graph may, in some embodiments, be embodied as a cloud service. For example, entities may integrate their clinical record processing system with the knowledge graph generation service and access the service as a Web service. In some embodiments, the knowledge graph generation service may be implemented as a Representational State Transfer Web Service (RESTful Web service).

Although FIG. 1 illustrates an example communication network including an intelligent DSS for associating medical entities in a knowledge graph for suggesting codes for one or more portions of a patient clinical record, it will be understood that embodiments of the inventive subject matter are not limited to such configurations, but are intended to encompass any configuration capable of carrying out the operations described herein.

FIG. 2 is a block diagram illustrating a multi-stage AI supported DSS 200 used in the knowledge graph generation server 140 and DSS module 145 of FIG. 1 in accordance with some embodiments of the disclosure. As shown in FIG. 2, the multi-stage AI supported DSS 200 includes a plurality of modules coupled in pipeline fashion. The multi-stage AI supported DSS 200 may be configured automate the operations involved in generating suggested medical entity relationship pairings based on one or more clinical records associated with patients and then incorporating the suggested medical entity pairings into a knowledge graph that can be used to configure a non-transitory computer readable medium. The multi-stage AI supported DSS 200 includes the following serially connected modules: an Optical Character Recognition (OCR) module 205 configured to convert the patient records into text records; a medical entity extraction model 210, which may embodied as a deep learning based NER model, that is configured to extract medical entities from clinical information included in one or more patient health records and/or medical claim information; a relationship strength module 215, which is configured to quantify the relationship strengths between pairs of medical entities based on a frequency of occurrence of the pairs in the clinical information; an uncommonality analysis module 220, which is configured to generate an uncommonality score for one or both of the medical entities in a pairing. The uncommonality score for a first medical entity in a pairing is indicative of a frequency that the first medical entity occurs with the second medical entity across an entire set of instances of the second medical entity in the clinical information. The AI supported DSS further includes a relevance score module 225, which is configured to generate a relevance score for each medical entity pairing. The relevance score, in some embodiments, is given by a product of the uncommonality score for a first one of the medical entities in a pairing and a log of the sum of one plus a number of times that the first medical entity occurs with a second medical entity in the pairing in the clinical information across the entire set of instances of the second medical entity in the clinical information. In other embodiments, the relevance score is given by a combination of a first product and a second product, where the first product is a product of the uncommonality score for the first one of the medical entities and a log of the sum of one plus a number of times that a first medical entity in the pairing occurs with a second medical entity in the pairing in the clinical information across the entire set of instances of the second medical entity in the clinical information and the second product is a product of the uncommonality score for the second one of the medical entities in the pairing and a log of the sum of one plus a number of times that the second medical entity occurs with the first medical entity in the pairing in the clinical information across the entire set of instances of the first medical entity in the clinical information. A knowledge graph generation module 230 is configured to configure a non-transitory computer readable medium with a knowledge graph representing those medical entity pairs having relevance scores that satisfy a relevance threshold.

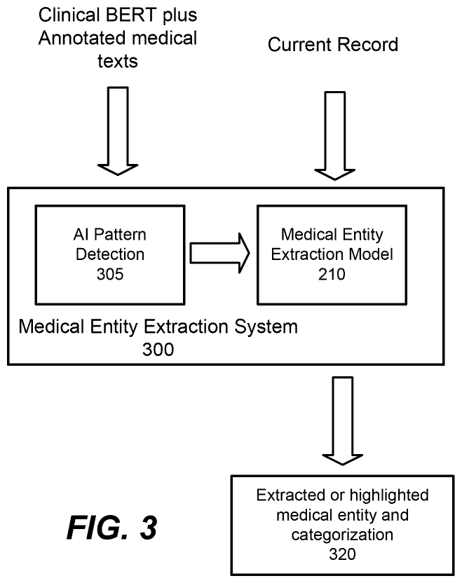

FIG. 3 is a block diagram of a medical entity extraction system 300 that may be used to provide the medical entity extraction model 210 of FIG. 2 in accordance with some embodiments of the disclosure. The medical extraction system 300 may be configured to generate a medical entity extraction model, which may be embodied as an NER model. NER is a form of NLP that involves extracting and identifying essential information from text. The information that is extracted and categorized is called an entity. It can be any word or a series of words that consistently refers to the same thing. According to some embodiments of the disclosure, the medical entity extraction system 300 is configured to classify named entities into the following pre-defined categories: disease (e.g., medical conditions and diagnoses), medication (e.g., pharmaceutical drugs or drug therapies used for treatment), procedure (e.g., diagnostic procedures, therapeutic procedures, and medical devices), lab (e.g., pathology and laboratory procedures), and symptom (e.g., clinical signs and symptoms). Within the medication category, the following sub-categories may be used: dosage (e.g., amount or strength of a drug including units), form (e.g., the physical form of a dose of drug when it is administered), and route (e.g., the way in which a drug is taken into the body). Different types of coding symptoms may map to different types of medical entity categories. The categories used classify the medical entities may span the different types of coding systems that are used to code the medical entities. The medical entity extraction system 300 includes an AI pattern detection module 305 and the medical entity extraction model 210. The AI pattern detection module 305 may be configured to receive, for example, a machine learning model, such as ClinicalBERT. BERT is a deep neural network that uses the transformer encoder architecture to learn embeddings for text. The transformer encoder architecture is based on a self-attention mechanism. ClinicalBERT is publicly available application of the BERT model to clinical information. The AI pattern detection module 305 may further train the ClinicalBERT model with annotated medical texts to generate the medical entity extraction model 210. During training, the AI pattern detection module 305 learns associations between names of objects in clinical text and relevant medical entities. Due to the non-standard usage of terms, abbreviations, synonyms, acronyms, and ambiguity in entity descriptions, a supervised deep learning based NER model is used to perform the medical entity extraction to improve the accuracy in identifying medical entities in clinical information, such as patient health records. The medical entity extraction model 210 may be configured to extract or highlight medical entities 320 contained in clinical information of on or more current records.

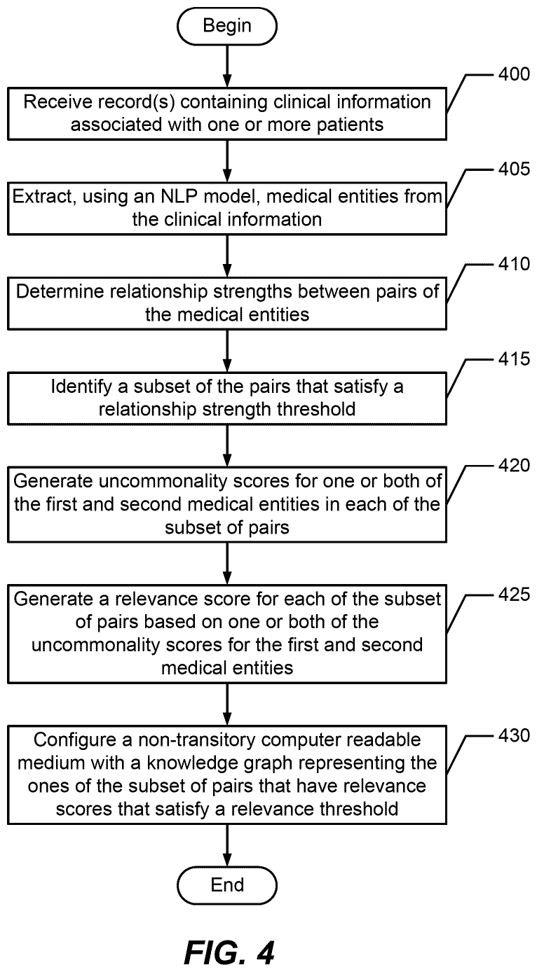

FIG. 4 is a flowchart that illustrates operations of the intelligent DSS for associating medical entities in a knowledge graph in accordance with some embodiments of the disclosure. Operations begin at block 400 where records containing clinical information associated with one or more patients is received. At block 405, the medical entity extraction model 210 is used to extract or highlight medical entities from the clinical information. The relationship strength module 215 is used to determine relationship strengths between pairs of the medical entities at block 410. In some embodiments, the relationship strengths between the medical entities are quantified based on a frequency of occurrence of the respective pairs in the clinical information. At block 415, those medical entity pairs that having relationship strengths that do not satisfy a relationship strength threshold may be discarded leaving a subset of medical entity pairs that may be candidates for inclusion in a medical entity knowledge graph. The uncommonality analysis module 220 generates uncommonality scores for one or both of the first and second medical entities in each of the subset of medical entity pairs at block 420. The uncommonality score for a first medical entity in a pairing is indicative of a frequency that the first medical entity occurs with the second medical entity across an entire set of instances of the second medical entity in the clinical information. Similarly, the uncommonality score for a second medical entity in a pairing is indicative of a frequency that the second medical entity occurs with the first medical entity across an entire set of instances of the first medical entity in the clinical information.

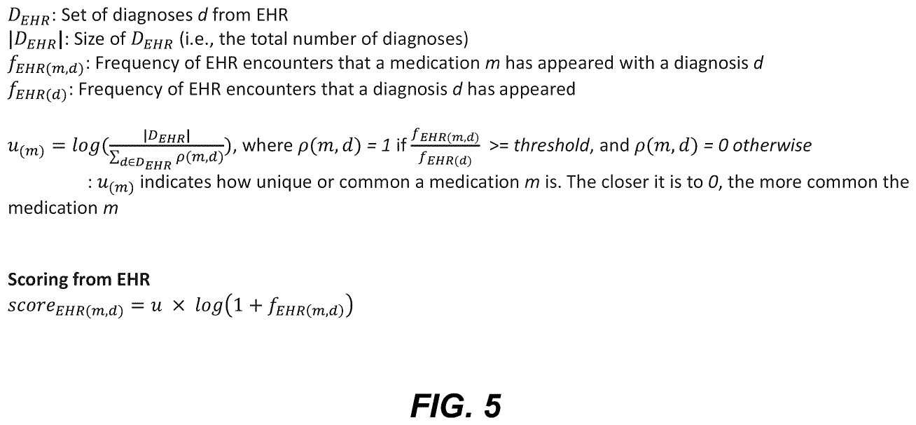

FIG. 5 illustrates generation of medical entity uncommonality scores in accordance with some embodiments of the disclosure. In the example of FIG. 5, an uncommonality score is generated for a first medical entity corresponding to a medication m that is paired with a second medical entity corresponding to a diagnosis d. As shown in FIG. 5, the uncommonality score u₍ₘ₎ for the medication m is given by a log of a ratio of a size of the set of distinct names that the diagnosis d can assume, i.e., the total number of diagnoses, to a sum across the entire set of instances of the diagnosis d in the clinical information of a first commonality factor ρ(m,d). The first commonality factor ρ(m,d) is equal to one when a ratio of a number of times that the medication m occurs with the diagnosis d in the clinical information across the entire set of instances of the diagnosis d in the clinical information to a number of times that the diagnosis d occurs in the clinical information satisfies a threshold and is zero otherwise.

Returning to FIG. 4, the relevance score module 225 generates a relevance score for each of the subset of pairs of medical entities at block 425 based on one or both of the uncommonality scores for the first and second medical entities in each pair. As shown in the FIG. 5 example, the relevance score_{EHR(m,d)} is given by a product of the uncommonality score for medication m in the pairing and a log of the sum of one plus a number of times that the medication m occurs with the diagnosis d in the pairing in the clinical information across the entire set of instances of the diagnosis d in the clinical information. FIG. 5 illustrates an example where the relevance score is based on the uncommonality of a single medical entity in a medical entity pairing. In other embodiments, the final relevance score may be based on a combination of a first relevance score based on a first one of the medical entities in the pairing and a second relevance score based on a second one of the medical entities in the pairings. That is, the final relevance score may be given by a combination of a first product and a second product, where the first product is a product of the uncommonality score for the first one of the medical entities and a log of the sum of one plus a number of times that a first medical entity in the pairing occurs with a second medical entity in the pairing in the clinical information across the entire set of instances of the second medical entity in the clinical information and the second product is a product of the uncommonality score for the second one of the medical entities in the pairing and a log of the sum of one plus a number of times that the second medical entity occurs with the first medical entity in the pairing in the clinical information across the entire set of instances of the first medical entity in the clinical information.

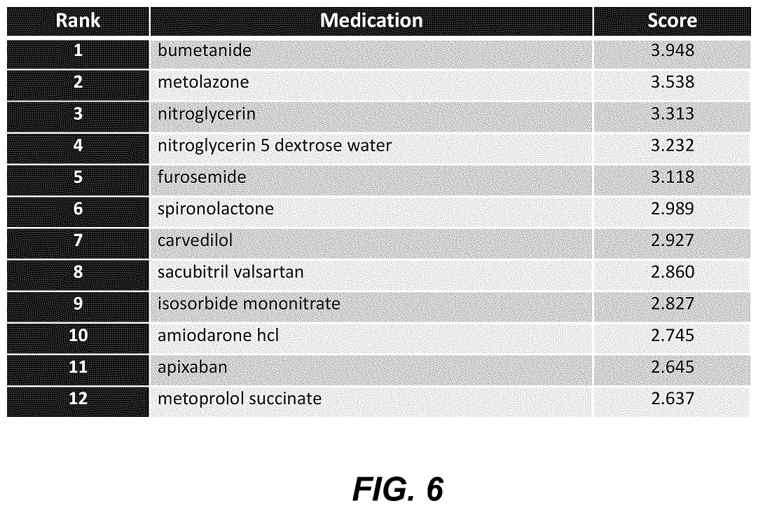

FIG. 6 is a table that illustrates ranking of medications based on a relevance score with respect to a diagnosis. In the example table shown in FIG. 6, twelve medications are listed and ranked based on their relevance scores when paired with a particular diagnosis taking into account the uncommonality analysis described above to lower the relevance score for those medications that may be unlikely to be associated with the diagnosis, but may nevertheless occur frequently in the clinical information.

Returning to FIG. 4, the knowledge graph generation module 230 configures a non-transitory computer readable medium with a knowledge graph at block 430 that represents the medical entity pairs in the subset of pairs identified based on relationship strength that have relevance scores that satisfy a relevance threshold. In some embodiments, the knowledge graph is embodied in the non-transitory computer readable medium using Resource Description Framework (RDF) triples, which are three positional statements. An RDF statement links resources using a uniform structure by identifying a subject predicate and object. In the knowledge graph, the nodes represent subjects and objects while the links represent predicates.

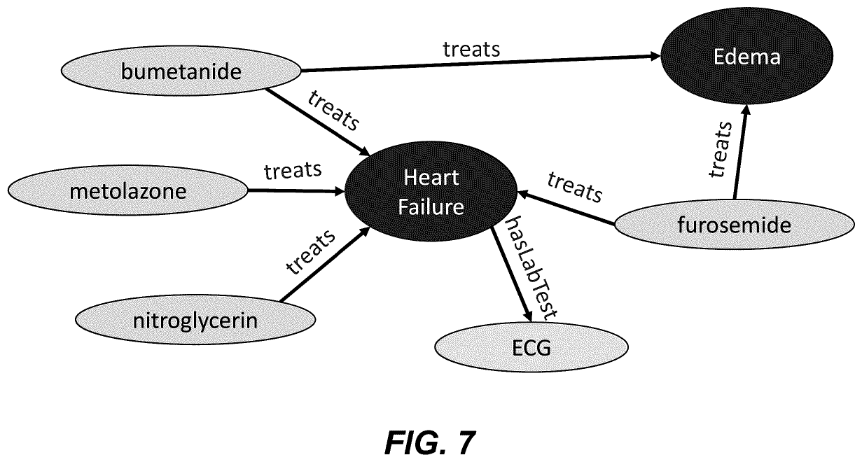

FIG. 7 is a knowledge graph generated using an intelligent DSS according to some embodiments of the disclosure. In the example of FIG. 7, drugs bumetanide, metolazone, nitroglycerin, and furosemide are shown as nodes along with diseases heart failure and edema. The edges used to connect the drugs to the nodes indicate that these drugs treat those diseases. An ECG is also listed as a node with an edge between the heart failure node and the ECG node to indicate that the ECG is a lab test for heart failure.

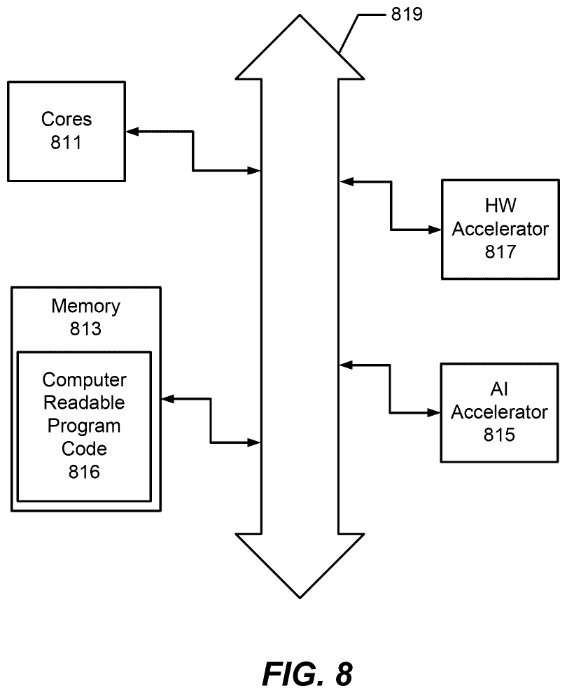

FIG. 8 is a block diagram of a data processing system that may be used to implement the knowledge graph generation server 140 of FIG. 1 and/or the medical entity extraction system 300 of FIG. 3 in accordance with some embodiments of the disclosure. As shown in FIG. 8, the data processing system may include at least one core 811, a memory 813, an artificial intelligence (AI) accelerator 815 and a hardware (HW) accelerator 817. The at least one core 811, the memory 813, the AI accelerator 815, and the HW accelerator 817 may communicate with each other through a bus 819.

The at least one core 811 may be configured to execute computer program instructions. For example, the at least one core 811 may execute an operating system and/or applications represented by the computer readable program code 816 stored in the memory 813. In some embodiments, the at least one core 811 may be configured to instruct the AI accelerator 815 and/or the HW accelerator 817 to perform operations by executing the instructions and obtain results of the operations from the AI accelerator 815 and/or the HW accelerator 817. In some embodiments, the at least one core 811 may be an ASIP customized for specific purposes and support a dedicated instruction set.

The memory 813 may have an arbitrary structure configured to store data. For example, the memory 813 may include a volatile memory device, such as dynamic random-access memory (DRAM) and static RAM (SRAM), or include a non-volatile memory device, such as flash memory and resistive RAM (RRAM). The at least one core 811, the AI accelerator 815, and the HW accelerator 817 may store data in the memory 813 or read data from the memory 813 through the bus 819.

The AI accelerator 815 may refer to hardware designed for AI applications. In some embodiments, the AI accelerator 815 may include one or more machine learning models configured to provide a DSS for associating medical entities in a knowledge graph. The AI accelerator 815 may generate output data by processing input data provided from the at least one core 815 and/or the HW accelerator 817 and provide the output data to the at least one core 811 and/or the HW accelerator 817. In some embodiments, the AI accelerator 815 may be programmable and be programmed by the at least one core 811 and/or the HW accelerator 817. The HW accelerator 817 may include hardware designed to perform specific operations at high speed. The HW accelerator 817 may be programmable and be programmed by the at least one core 811.

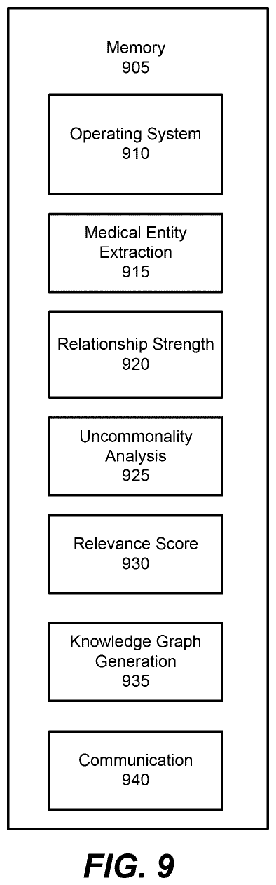

FIG. 9 illustrates a memory 905 that may be used in embodiments of data processing systems, such as the knowledge graph generation server 140 of FIG. 1, the medical entity extraction system 300, and the data processing system of FIG. 8, respectively, to provide an AI supplemented DSS for associating medical entities in a knowledge graph. The memory 905 is representative of the one or more memory devices containing the software and data used for facilitating operations of the knowledge graph generation server 140 and the DSS module 145 as described herein. The memory 905 may include, but is not limited to, the following types of devices: cache, ROM, PROM, EPROM, EEPROM, flash, SRAM, and DRAM. As shown in FIG. 9, the memory 905 may contain seven or more categories of software and/or data: an operating system 910, a medical entity extraction module 915, a relationship strength module 920, an uncommonality analysis module 925, a relevance score module 930, a knowledge graph generation module 935, and a communication module 940. In particular, the operating system 910 may manage the data processing system's software and/or hardware resources and may coordinate execution of programs by the processor.

The medical entity extraction module 915 may be configured to perform one or more of the operations described above with respect to the medical entity extraction system 300 of FIG. 3 and the flowchart of FIG. 4. The relationship strength module 920 may be configured to perform one or more of the operations described above with respect to the relationship strength module 215 of FIG. 2 and the flowchart of FIG. 4. The uncommonality analysis module 925 may be configured to perform one or more of the operations described above with respect to the uncommonality analysis module 220 of FIG. 2 and the flowchart of FIG. 4. The relevance score module 930 may be configured to perform one or more of the operations described above with respect to the relevance score module 225 of FIG. 2 and the flowchart of FIG. 4. The knowledge graph generation module 230 may be configured to perform one or more of the operations described above with respect to the knowledge graph generation module 230 of FIG. 2 and the flowchart of FIG. 4.

Although FIGS. 8 and 9 illustrate hardware/software architectures that may be used in data processing systems, such as the knowledge graph generation server 140 of FIG. 1, the medical entity extraction system 300 and the data processing system of FIG. 8, respectively, in accordance with some embodiments of the disclosure, it will be understood that embodiments of the disclosure are not limited to such a configuration but is intended to encompass any configuration capable of carrying out operations described herein.

Computer program code for carrying out operations of data processing systems discussed above with respect to FIGS. 1-9 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the intermediary server 130 of FIG. 1, the knowledge graph generation server 140 of FIG. 1, the medical entity extraction system 300, and the data processing system of FIG. 8 may each be implemented as a single processor system, a multi-processor system, a multi-core processor system, or even a network of stand-alone computer systems, in accordance with various embodiments of the disclosure. Each of these processor/computer systems may be referred to as a “processor” or “data processing system.” The functionality provided by the intermediary server 130 and the knowledge graph generation server 140 may be merged into a single server or maintained as separate servers in accordance with different embodiments of the disclosure.

The data processing apparatus described herein with respect to FIGS. 1-9 may be used to facilitate providing an AI supplemented DSS for associating medical entities in a knowledge graph according to some embodiments of the disclosure described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 905 when coupled to a processor includes computer readable program code that, when executed by the processor, causes the processor to perform operations including one or more of the operations described herein with respect to FIGS. 1-9.

Advantageously, the DSS for associating medical entities in a knowledge graph may provide a compilation of health care information in a knowledge graph database format that can be efficiently accessed using one or more computers to reduce processor time in gathering the same or similar data and drawing the same types of inferences between such medical entities. Moreover, the database format in which the knowledge graph is stored in the memory makes efficient use of the memory thereby saving memory resources. Moreover, the accuracy of the knowledge graph relationships between medical entities inferred using embodiments of the DSS described herein may rival that of knowledge graphs manually generated using medical professionals.
Some embodiments of the disclosure may provide an intelligence decision support system as set forth by the following examples: Example 1: a computer-implemented method comprises: receiving, by one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.
Example 2: the computer-implemented method of Example 1, wherein the NLP model is a deep learning model.
Example 3: the computer-implemented method of Examples 1 and 2, wherein determining the relationship strengths comprises: quantifying, by the one or more processors, the relationship strengths between the pairs of respective ones of the medical entities based on a frequency of occurrence of respective ones of the pairs in the clinical information.
Example 4: the computer-implemented method of any of Examples 1-3, wherein the uncommonality score for the first one of medical entities is given by a log of a ratio of a size of the set of distinct names that the second medical entity can assume to a sum across the entire set of instances of the second medical entity in the clinical information of a first commonality factor; and the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.
Example 5: the computer-implemented method of Example 4, wherein the uncommonality score for the second one of medical entities is given by a log of a ratio of a size of the set of distinct names that the first medical entity can assume to a sum across the entire set of instances of the first medical entity in the clinical information of a second commonality factor; and the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.
Example 6: the computer-implemented method of any of Examples 1-5, wherein the relevance score is given by a product of the uncommonality score for the first one of the medical entities and a log of the sum of one plus a number of times that the first medical entity occurs with the second medical entity in the clinical information across the entire set of instances of the second medical entity in the clinical information.
Example 7: the computer-implemented method of Examples 1-5, wherein the relevance score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.
Example 8: the computer-implemented method of any of Examples 1-7, wherein generating the knowledge graph comprises: generating, by the one or more processors, Resource Description Framework (RDF) triples based on the subset of pairs having relevance scores, respectively, that satisfy the relevance threshold; and configuring, by the one or more processors, the knowledge graph with the RDF triples.
Example 9: the computer-implemented method of any of Examples 1-8, wherein the clinical information comprises patient health record information, medical claim information, or both the patient health record information and the medical claim information.
Example 10: a system comprises one or more processors and a memory coupled to the one or more processors and comprising computer readable program code embodied in the memory that is executable by the processor to perform operations comprising: receiving, by the one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.
Example 11: the system of Example 10, wherein the NLP model is a deep learning model.
Example 12: the system of any of Examples 10 and 11, wherein determining the relationship strengths comprises: quantifying, by the one or more processors, the relationship strengths between the pairs of respective ones of the medical entities based on a frequency of occurrence of respective ones of the pairs in the clinical information.
Example 13: the system of any of Examples 10-12, wherein the uncommonality score for the first one of medical entities is given by a log of a ratio of a size of the set of distinct names that the second medical entity can assume to a sum across the entire set of instances of the second medical entity in the clinical information of a first commonality factor; and the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.
Example 14: the system of Example 13, wherein the uncommonality score for the second one of medical entities is given by a log of a ratio of a size of the set of distinct names that the first medical entity can assume to a sum across the entire set of instances of the first medical entity in the clinical information of a second commonality factor; and the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.
Example 15: the system of any of Examples 10-14, wherein the relevance score is given by a product of the uncommonality score for the first one of the medical entities and a log of the sum of one plus a number of times that the first medical entity occurs with the second medical entity in the clinical information across the entire set of instances of the second medical entity in the clinical information.
Example 16: the system of any of Examples 10-14 wherein the relevance

score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.
Example 17: the system of any of Examples 10-16, wherein generating the knowledge graph comprises: generating, by the one or more processors, Resource Description Framework (RDF) triples based on the subset of pairs having relevance scores, respectively, that satisfy the relevance threshold; and configuring, by the one or more processors, the knowledge graph with the RDF triples.
Example 18: the system of any of Examples 10-17, wherein the clinical information comprises patient health record information, medical claim information, or both the patient health record information and the medical claim information.
Example 19: One or more a non-transitory computer readable storage media comprise computer readable program code embodied in the media that is executable by one or more processors to perform operations comprising: receiving, by one or more processors, a plurality of records containing clinical information associated with one or more patients; extracting, using a Natural Language Processing (NLP) model and the one or more processors, a plurality of medical entities from the clinical information to generate a first data set that contains the plurality of medical entities; denoising, by the one or more processors, the first dataset to generate a second data set by: determining, by the one or more processors, relationship strengths between pairs of respective ones of the medical entities; identifying, by the one or more processors, a subset of the pairs of the respective ones of the plurality of medical entities that satisfy a relationship strength threshold; generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and generating, by the one or more processors, a relevance score for each of the subset of pairs based on one or both of the uncommonality scores for the first and second ones of the medical entities included in the respective pair and a frequency of occurrence of the respective pair in the clinical information; and generating, by the one or more processors, a knowledge graph data structure representing ones of the subset of pairs having relevance scores, respectively, that satisfy a relevance threshold.
Example 20: the computer-program product of Example 19, wherein the relevance score is given by a combination of a first product and a second product; the first product is a product of the uncommonality score for the first one of the medical entities and a log of the sum of one plus a number of times that the first medical entity occurs with the second medical entity in the clinical information across the entire set of instances of the second medical entity in the clinical information; and the second product is a product of the uncommonality score for the second one of the medical entities and a log of the sum of one plus a number of times that the second medical entity occurs with the first medical entity in the clinical information across the entire set of instances of the first medical entity in the clinical information.

### Further Definitions and Embodiments

In the above-description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the embodiments of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the embodiments of the present disclosure has been presented for purposes of illustration and description, but is not intended to be exhaustive or limited to the embodiments in the form disclosed. Many modifications and variations will be apparent to those of ordinary skill in the art without departing from the scope and spirit of the disclosed embodiments. The aspects of the disclosure herein were chosen and described to best explain the principles of the embodiments and the practical application, and to enable others of ordinary skill in the art to understand the embodiments with various modifications as are suited to the particular use contemplated.

What is claimed is:

1. A computer-implemented method, comprising:
receiving, by one or more processors, a plurality of records containing clinical information associated with one or more patients;
extracting, using a Natural Language Processing (NLP) model and the one or more processors, a plurality of medical entities from the clinical information to generate a first data set that contains the plurality of medical entities;
denoising, by the one or more processors, the first dataset to generate a second data set by:
determining, by the one or more processors, relationship strengths between pairs of respective ones of the medical entities;
identifying, by the one or more processors, a subset of the pairs of the respective ones of the plurality of medical entities that satisfy a relationship strength threshold;
generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and
generating, by the one or more processors, a relevance score for each of the subset of pairs based on one or both of the uncommonality scores for the first and second ones of the medical entities included in the respective pair and a frequency of occurrence of the respective pair in the clinical information; and
generating, by the one or more processors, a knowledge graph data structure representing ones of the subset of pairs having relevance scores, respectively, that satisfy a relevance threshold.

2. The computer-implemented method of claim 1, wherein the NLP model is a deep learning model.

3. The computer-implemented method of claim 1, wherein determining the relationship strengths comprises:
quantifying, by the one or more processors, the relationship strengths between the pairs of respective ones of the medical entities based on a frequency of occurrence of respective ones of the pairs in the clinical information.

4. The computer-implemented method of claim 1, wherein the uncommonality score for the first one of medical entities is given by a log of a ratio of a size of the set of distinct names that the second medical entity can assume to a sum across the entire set of instances of the second medical entity in the clinical information of a first commonality factor; and
wherein the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.

5. The computer-implemented method of claim 4, wherein the uncommonality score for the second one of medical entities is given by a log of a ratio of a size of the set of distinct names that the first medical entity can assume to a sum across the entire set of instances of the first medical entity in the clinical information of a second commonality factor; and
wherein the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.

6. The computer-implemented method of claim 1, wherein the relevance score is given by a product of the uncommonality score for the first one of the medical entities and a log of the sum of one plus a number of times that the first medical entity occurs with the second medical entity in the clinical information across the entire set of instances of the second medical entity in the clinical information.

7. The computer-implemented method of claim 1, wherein the relevance score is given by a combination of a first product and a second product;
wherein the first product is a product of the uncommonality score for the first one of the medical entities and a log of the sum of one plus a number of times that the first medical entity occurs with the second medical entity in the clinical information across the entire set of instances of the second medical entity in the clinical information; and
wherein the second product is a product of the uncommonality score for the second one of the medical entities and a log of the sum of one plus a number of times that the second medical entity occurs with the first medical entity in the clinical information across the entire set of instances of the first medical entity in the clinical information.

8. The computer-implemented method of claim 1, wherein generating the knowledge graph comprises:
generating, by the one or more processors, Resource Description Framework (RDF) triples based on the subset of pairs having relevance scores, respectively, that satisfy the relevance threshold; and
configuring, by the one or more processors, the knowledge graph with the RDF triples.

9. The computer-implemented method of claim 1, wherein the clinical information comprises patient health record information, medical claim information, or both the patient health record information and the medical claim information.

10. A system, comprising:
one or more processors; and
a memory coupled to the one or more processors and comprising computer readable program code embodied in the memory that is executable by the one or more processors to perform operations comprising:
receiving, by the one or more processors, a plurality of records containing clinical information associated with one or more patients;
extracting, using a Natural Language Processing (NLP) model and the one or more processors, a plurality of medical entities from the clinical information to generate a first data set that contains the plurality of medical entities;
denoising, by the one or more processors, the first dataset to generate a second data set by:
determining, by the one or more processors, relationship strengths between pairs of respective ones of the medical entities;
identifying, by the one or more processors, a subset of the pairs of the respective ones of the plurality of medical entities that satisfy a relationship strength threshold;
generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and
generating, by the one or more processors, a relevance score for each of the subset of pairs based on one or both of the uncommonality scores for the first and second ones of the medical entities included in the respective pair and a frequency of occurrence of the respective pair in the clinical information; and
generating, by the one or more processors, a knowledge graph data structure representing ones of the subset of pairs having relevance scores, respectively, that satisfy a relevance threshold.

11. The system of claim 10, wherein the NLP model is a deep learning model.

12. The system of claim 10, wherein determining the relationship strengths comprises:
quantifying, by the one or more processors, the relationship strengths between the pairs of respective ones of the medical entities based on a frequency of occurrence of respective ones of the pairs in the clinical information.

13. The system of claim 10, wherein the uncommonality score for the first one of medical entities is given by a log of a ratio of a size of the set of distinct names that the second medical entity can assume to a sum across the entire set of instances of the second medical entity in the clinical information of a first commonality factor; and
wherein the first commonality factor is equal to one when a ratio of a number of times that the first medical entity occurs with the second medical entity in the clinical information across the entire set of instances of the second medical entity in the clinical information to a number of times that the second medical entity occurs in the clinical information satisfies a threshold and is zero otherwise.

14. The system of claim 13, wherein the uncommonality score for the second one of medical entities is given by a log of a ratio of a size of the set of distinct names that the first medical entity can assume to a sum across the entire set of instances of the first medical entity in the clinical information of a second commonality factor; and
wherein the second commonality factor is equal to one when a ratio of a number of times that the second medical entity occurs with the first medical entity in the clinical information across the entire set of instances of the first medical entity in the clinical information to a number of times that the first medical entity occurs in the clinical information satisfies a threshold and is zero otherwise.

15. The system of claim 10, wherein the relevance score is given by a product of the uncommonality score for the first one of the medical entities and a log of the sum of one plus a number of times that the first medical entity occurs with the second medical entity in the clinical information across the entire set of instances of the second medical entity in the clinical information.

16. The system of claim 10, wherein the relevance score is given by a combination of a first product and a second product;
wherein the first product is a product of the uncommonality score for the first one of the medical entities and a log of the sum of one plus a number of times that the first medical entity occurs with the second medical entity in the clinical information across the entire set of instances of the second medical entity in the clinical information; and
wherein the second product is a product of the uncommonality score for the second one of the medical entities and a log of the sum of one plus a number of times that the second medical entity occurs with the first medical entity in the clinical information across the entire set of instances of the first medical entity in the clinical information.

17. The system of claim 10, wherein generating the knowledge graph comprises:
generating, by the one or more processors, Resource Description Framework (RDF) triples based on the subset of pairs having relevance scores, respectively, that satisfy the relevance threshold; and
configuring, by the one or more processors, the knowledge graph with the RDF triples.

18. The system of claim 10, wherein the clinical information comprises patient health record information, medical claim information, or both the patient health record information and the medical claim information.

19. One or more a non-transitory computer readable storage media comprise computer readable program code embodied in the media that is executable by one or more processors to perform operations comprising:
receiving, by the one or more processors, a plurality of records containing clinical information associated with one or more patients;
extracting, using a Natural Language Processing (NLP) model and the one or more processors, a plurality of medical entities from the clinical information to generate a first data set that contains the plurality of medical entities;
denoising, by the one or more processors, the first dataset to generate a second data set by:
determining, by the one or more processors, relationship strengths between pairs of respective ones of the medical entities;
identifying, by the one or more processors, a subset of the pairs of the respective ones of the plurality of medical entities that satisfy a relationship strength threshold;
generating, by the one or more processors, uncommonality scores for one or both of a first and a second medical entity in each of the subset of pairs, the uncommonality score for the first medical entity being indicative of a frequency that the first medical entity occurs with the second medical entity across an entire set of instances of the second medical entity in the clinical information, the uncommonality score for the second medical entity being indicative of a frequency that the second medical entity occurs with the first medical entity across an entire set of instances of the first medical entity in the clinical information; and
generating, by the one or more processors, a relevance score for each of the subset of pairs based on one or both of the uncommonality scores for the first and second ones of the medical entities included in the respective pair and a frequency of occurrence of the respective pair in the clinical information; and
generating, by the one or more processors, a knowledge graph data structure representing ones of the subset of pairs having relevance scores, respectively, that satisfy a relevance threshold.

20. The non-transitory computer readable storage media of claim 19, wherein the relevance score is given by a combination of a first product and a second product;
wherein the first product is a product of the uncommonality score for the first one of the medical entities and a log of the sum of one plus a number of times that the first medical entity occurs with the second medical entity in the clinical information across the entire set of instances of the second medical entity in the clinical information; and
wherein the second product is a product of the uncommonality score for the second one of the medical entities and a log of the sum of one plus a number of times that the second medical entity occurs with the first medical entity in the clinical information across the entire set of instances of the first medical entity in the clinical information.
